(19) United States
(12) Patent Application Publication
(10) Pub. No.: US 2003/0200999 A1
(43) Pub. Date: Oct. 30, 2003

(54) Method and apparatus for cleansing skin

(76) Inventor: Arthur F. Clark, Golden, CO (US)

Correspondence Address: CLARK & BRODY, Suite 600, 1750 K Street, NW, Washington, DC, 20006, US

(21) Appl. No.: 10/424,851

(22) Filed: Apr. 29, 2003

Related U.S. Application Data
(60) Provisional application No. 60/375,836, filed on Apr. 29, 2002.

(51) Int. Cl. B08B 1/02

(52) U.S. Cl. 134/32; 134/34

(57) ABSTRACT

A device for cleansing skin includes a small cup forming a cavity and a spray nozzle connected to a source of cleansing fluid. The cup is easily manipulated to place the edge of the cup on the surface of skin to be cleansed so that the nozzle is aimed at the skin. The cup prevents splattering of the fluid. A drain line may be included in the cup to drain spent washing fluids.

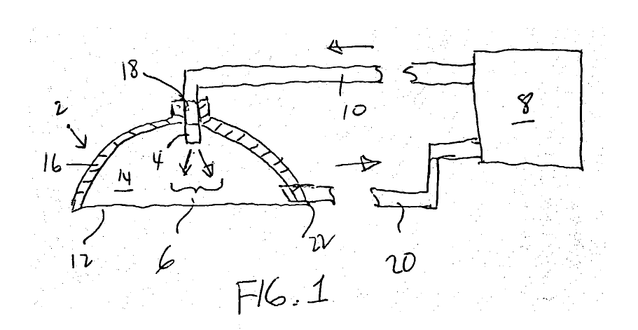

### CROSS REFERENCE TO RELATED APPLICATION

This application claims the benefit of provisional application serial No. 60/375,836, which was filed on Apr. 29, 2002.

### TECHNICAL FIELD

This invention relates to the art of devices used for cleansing skin, particularly the skin on one's face.

### BACKGROUND

It is often desirable to clean one's skin thoroughly without rubbing it with an article such as a washcloth. This is because a washcloth often abrades the skin undesirably and because a washcloth can actually be a source of bacteria itself. A preferable method of washing delicate or infected skin, or tissue, is by irrigating the skin with a cleansing fluid under low pressure. Apparatus providing this feature are, however, not available outside the clinical setting.

### SUMMARY OF THE INVENTION

The invention provides an inexpensive and easily available device for cleansing skin with a stream of cleansing fluid. The device includes a small, light hand-held cleansing cup that is easily applied to the skin to be cleansed, such as the face and moved about over the surface to effect cleansing. The cup forms a cavity that is preferably a portion of a sphere. The bottom of the cup terminates in an edge forming an opening. When the edge is placed in contact with the surface of the skin to be cleaned, the cavity communicates with the skin through the opening.

The upper part of the cup receives a nozzle that is configured to direct a stream of cleansing fluid into the cavity toward the opening so that the fluid stream is incident on the skin to be cleaned when the bottom edge of the cleansing cup is in contact, or approximately in contact, with the skin to be cleansed. The cleansing fluid then irrigates the skin to be cleansed, while the cup catches any fluid that may splash from the surface of the skin. This allows the cleansing process to be accomplished with a minimum of splattering of the cleansing fluid.

In one embodiment, the waste cleansing fluid is allowed to escape through a gap between the bottom edge and the skin. For example, if one is washing one's face over a sink, the cup is simply lifted from the surface of the face periodically so that the waste fluid is collected in the sink.

In a second embodiment, the cup is provided with one or more return lines that drain the waste fluid to a remote reservoir. Alternatively, the return line is in communication with a receptacle such as a sink, and the return line is simply a drain. Thus, the used washing fluid can be either discarded or reused.

Any of several sources of cleansing fluid are useful with the invention. For example, a fluid line can be attached to a common spigot found at a bathroom sink, and a nozzle may be provided on the fluid line and received in the cleansing cup. Preferably, however, the source of cleansing fluid is an electric pump of the type sold by Water Pik, which includes a reservoir for cleansing fluids. In this later situation, cleansing chemicals may be added to the reservoir to enhance the effectiveness of the washing.

The cleansing fluid is preferably a liquid, but the invention contemplates that it is a fluid, a fluid mixed with a gas, or a gas alone. The gas may be air or a disinfectant gas. The liquid may be water or water with soap or disinfectant or antibiotic or the like. The fluid may also contain particles, such as pumice or sand and may be a slurry comprising a liquid with such particles entrained therein.

While the preferred embodiment utilizes a source of cleansing fluid under pressure, it is contemplated that the cup may be subjected to a vacuum such that the cleansing fluid is drawn into the cup from a reservoir at atmospheric pressure. For example, the return line may be connected to a vacuum source to withdraw the used fluid and to pull the cleansing fluid toward the cup. In that case the cleansing fluid may also be under pressure, in which case the vacuum assists the flow of cleansing fluid, or be at atmospheric pressure, in which case the vacuum is the sole cause of the flow. If the cup is subjected to a vacuum, either through the return line or another connection, a hole may be provided for receiving the user's finger to adjust or control the level of vacuum in the cup.

The nozzle may be any of several shapes depending on the desired flow rate and spray pattern. Thus, the nozzle could create a direct stream of cleansing fluid or it could create a spray pattern. The nozzles can be dedicated to a specific pattern or can be adjustable. In a most preferred embodiment, the cleansing cup is configured to receive a know nozzle from a Water Pik system so that it may be easily detached. This allows the cup to be washed independently and also allows the Water Pik to be used for its usual purpose of cleaning teeth.

### BRIEF DESCRIPTION OF THE DRAWINGS

The figure is a schematic of a skin cleansing system in accordance with the invention showing a vertical cross section of one embodiment of the cleansing cup.

### DETAILED DESCRIPTION OF THE PREFERRED EMBODIMENT

The drawing figure illustrates a skin cleansing system having a cleansing cup 2 that supports a nozzle 4 for applying a stream 6 of cleansing fluid to skin, such as facial skin, during cleansing. The nozzle is connected to a source 8 of cleansing fluid under pressure. A supply hose 10 connects the source 8 to the nozzle 4. The source 8, hose 10, and nozzle 4 may comprise a known system sold under the trademark Water Pik and used for cleaning teeth in the home environment.

The cleansing cup 2 is preferably spherical but may have other shapes. The bottom edge 12 of the cup forms an opening to a cavity 14 formed by the cup. This edge is preferably rounded so that it can comfortably engage the surface of the skin to the cleansed. The wall 16 of the cup is preferably thin and somewhat flexible so that the shape of the cup can conform to the shape of the surface to be cleansed. For example, known silicone materials will be useful. As well, however, the cup may be made of a stiffer material but with more flexible parts. For example, the upper part of the cup may be made of one material while the bottom part having the edge 12 is more flexible for conforming to the shapes of different parts of the user's face or other part of the body being cleansed.

The upper part of the cup includes an opening for receiving the nozzle 4 in such a manner that it is can be removed to allow the cup to be washed and/or sterilized. Preferably the opening slidingly receives the nozzle of the Water-Pik. Other connections may be used, including bayonet style or threaded connections.

It will be appreciated that the cup prevents splattering of the cleansing fluid from the skin being cleansed. In one embodiment, the waste cleansing fluid is simply allowed to escape between the edge 12 and the skin and to fall into a sink or bathtub. In another embodiment, the source 8 of fluid is provided with a return line 20 for the waste fluid. This return line may be connected to the cup through a second opening 22 near the edge 12. Of course, multiple return lines may be provided even though only one is illustrated.

In use, the user simply attaches the nozzle 4 to the cup 2, places a desired cleansing fluid in the reservoir of the source 8, and activates the source. The user holds the cup adjacent the skin to be cleansed so that the stream 6 of cleansing fluid is incident on the skin and moves it about to cleanse the entire surface.

The source 8 may provide several features such as adjustable flow rates, pressures, or pulse modes of the cleansing stream. Preferably a pulsed, low-pressure stream is provided.

The outlet line 10 may be provided with a handle and control elements, such as a control valve or controls for setting the pressure, flow rate or pulse mode. As well, the line 20 may be attached to the line 10 or a dual lumen tube may be provided.

While the nozzle 4, return line 20, and cup have been shown as separate elements, it is contemplated that these be combined into an integral unit.

The nozzle is shown in the figure as centrally located in the cup, but it may be placed at other locations nearer one side of the cup. As well, the orientation of the nozzle shown in generally radial for a spherical cup, but it may have other orientations. For example, it may be desirable to orient the nozzle such that it directs the stream of cleansing fluid toward the opening in a non-perpendicular direction. As well, the cup may be provided with an adjustable mounting so that the orientation of the nozzle can be changed either before or during use.

Modifications within the scope of the appended claims will be apparent to those of skill in the art.

I claim:

1. Apparatus for cleansing skin comprising a cleansing cup and a nozzle for receiving a stream of cleansing fluid, wherein said cleansing cup forms a cavity having an edge forming an opening, and said nozzle is oriented to direct said cleansing fluid into said cavity toward said opening.

2. Apparatus according to claim 1 wherein said cleansing cup removably receives said nozzle.

3. Apparatus according to claim 1 further comprising a source of said stream of cleansing fluid.

4. Apparatus according to claim 1 wherein said cleansing cup is configured to receive a return line for waste cleansing fluid.

5. A method for cleansing skin comprising:
providing a source of a stream of cleansing fluid to a nozzle,
providing a cleansing cup that forms a cavity with an edge forming an opening,
attaching said nozzle to said cup such that said nozzle directs said cleansing fluid into said cavity toward said opening, and
applying said edge to the surface of skin to be cleansed.
